(19) United States
(12) Patent Application Publication
(10) Pub. No.: US 2015/0359866 A1
(43) Pub. Date: Dec. 17, 2015

(54) TREATMENT OF CERVICAL AND RELATED NEOPLASIAS WITH TOPICAL IMMUNOMODULATORS

(71) Applicant: THE JOHNS HOPKINS UNIVERSITY, Baltimore, MD (US)

(72) Inventor: Cornelia L. Trimble, Baltimore, MD (US)

(21) Appl. No.: 14/737,526

(22) Filed: Jun. 12, 2015

Related U.S. Application Data
(60) Provisional application No. 62/011,849, filed on Jun. 13, 2014.

(51) Int. Cl. A61K 39/00

(52) U.S. Cl. CPC A61K 39/0011

(57) ABSTRACT

The present invention provides methods for treating cervical and related HPV-associated neoplasias in subjects who are HPV16 negative comprising administering to the patient an effective amount of an immunomodulator, such as a Toll-like receptor agonist to the site of the lesions two or more times over a period of weeks to achieve regression of the neoplasia in the patient. Methods comprising the use of the immunomodulator with additional biological agents are also provided.

### REFERENCE TO RELATED APPLICATIONS

This application claims the benefit of U.S. Provisional Patent Application No. 62/011,849, filed on Jun. 13, 2014, which is hereby incorporated by reference for all purposes as if fully set forth herein.

### STATEMENT OF GOVERNMENTAL INTEREST

This invention was made with U.S. government support under grant nos. 5P30-CA6973, R01 CA142691, and P50 CA098252 from the National Institutes of Health. The U.S. government has certain rights in the invention.

### BACKGROUND OF THE INVENTION

Human papillomaviruses (HPVs) are the primary etiologic agents for cervical cancer and subsets of vaginal, vulvar and anal cancers and head and neck cancers. Approximately 90% of vaginal, vulvar and anal cancers associated with HPV are attributable to HPV16. However, 40% of high grade cervical intraepithelial neoplasias (CIN2/3) are caused by other non-HPV16 genotypes.

It has been demonstrated that a subset of CIN2/3 can undergo complete regression, within a relatively short timeframe. However, lesions caused by HPV16 are less likely to undergo spontaneous regression than lesions caused by other HPV types. Since there is no standardized way to distinguish lesions that are likely to regress, all women with a diagnosis of CIN2/3 undergo resection. While surgical treatment is effective and well tolerated for pre-cancer and early cancer lesions of the cervix, a subset of treated patients are at risk for preterm delivery and/or cervical incompetence, and premature rupture of membrane, which can be devastating to young women. Furthermore, surgical treatment of vaginal, vulvar and anal intraepithelial lesions is associated with significant morbidity and high recurrence rates. As such, it is important to develop effective alternative therapeutic methodologies to treat HPV-associated neoplasias, particularly for patients who are HPV16 negative.

In addition to surgical methods, application of an immunomodulator locally can enhance site-specific immunity. For example, Luci et al linked the nontoxic B subunit of cholera toxin (CTB) to model antigen OVA and found that its administration intravaginally increased OVA-specific CD8+ T cells in the draining lymph nodes and genital mucosa (J Immuno, 2006; 176:2749-57). Additionally, Wille-Reece et al found that vaccination of non-human primates using toll-like receptor (TLR) agonists as adjuvants generated stable antigen-specific CD8+ T cells (J. Exp. Med., 2006; 203:1249-58).

As such, there still exists a need for improved non-surgical methods for treating HPV-associated neoplasias in patients who are HPV16 negative.

### SUMMARY OF THE INVENTION

In accordance with an embodiment, the present invention provides a treatment regimen for generating an immune response against HPV-associated neoplasia in a subject that is HPV16 negative, comprising administering to the subject an effective amount of a composition comprising an immunomodulator to the site of the HPV-associated neoplasia in the subject.

In accordance with another embodiment, the present invention provides a treatment regimen for generating an immune response against HPV-associated neoplasia in a subject that is HPV16 negative, comprising administering to the subject an effective amount of a composition comprising an immunomodulator that is a toll-like receptor (TLR) ligand to the site of the HPV-associated neoplasia in the subject.

In accordance with a further embodiment, the present invention provides a treatment regimen for generating an immune response against HPV-associated neoplasia in a subject that is HPV16 negative, wherein regimen comprises a) topical administration of a sufficient amount of the immunomodulator to the site of the HPV-associated neoplasia in the subject at Day 0; b) repeating administration of a sufficient amount of the immunomodulator to the site of the HPV-associated neoplasia in the subject at an interval of about 3 to 5 weeks later; and c) repeating administration, at least one additional time, of a sufficient amount of the immunomodulator to the site of the HPV-associated neoplasia in the subject at an interval of about 3 to 5 weeks later.

### BRIEF DESCRIPTION OF THE DRAWINGS

FIG. 1: Direct ex vivo phenotypic analysis of cervical lymphocytes demonstrates that antigen-experienced T cells predominate in this mucosal compartment: discrete CD4, CD8, and CD45RO populations are identified.

FIG. 2: FACs of cervical lymphocytes from a vaccinated patient who did not regress her lesion: 22% of CD4+ lymphocytes also express Foxp3.

FIG. 3: Comparison of frequency of Treg cells in peripheral blood and cervical lymphocyte specimens in unvaccinated patients (same time points).

FIG. 4 shows data from clinical trial depicting the unexpected regression rate of patients with CIN2/3 that were HPV16 negative and treated with imiquimod. The rate of regression in the same timeframe of patients with CIN2/3 that were HPV16 negative without any other vaccine administration or imiquimod was 30-40%, whereas the rate of regression of patients with CIN2/3 that were HPV16 negative that had imiquimod treatment was nearly double the untreated patients.

### DETAILED DESCRIPTION OF THE INVENTION

The human papillomavirus is a DNA tumor virus that causes epithelial proliferation at cutaneous and mucosal surfaces. More than 100 different types of the virus exist, including approximately 30 to 40 strains that infect the human genital tract. Of these, there are oncogenic or high-risk types (16, 18, 31, 33, 35, 39, 45, 51, 52, and 58) that are associated with cervical, vulvar, vaginal, penile, oral, throat and anal cancers, and non-oncogenic or low-risk types (6, 11, 40, 42, 43, 44, and 54) that are associated with anogenital condyloma or genital warts. HPV 16 is the most oncogenic, accounting for almost half of all cervical cancers, and HPV 16 and 18 together account for approximately 70% of cervical cancers. HPV 6 and 11 are the most common strains associated with genital warts and are responsible for approximately 90% of these lesions.

The inventive methods show that lesional mechanisms of immune suppression can be manipulated. Use of an immunomodulator, such as a topical Toll-like receptor (TLR) agonist, at the site of the lesion will aid in identification of mechanisms of local immune inhibition will provide specific targets for manipulation that may enable effective anti-HPV immunity.

In accordance with an embodiment, the HPV-associated neoplasias treated by the inventive methods include cervical intraepithelial neoplasia (CIN), vulvar intraepithelial neoplasia (VIN), and vaginal intraepithelial neoplasia (VAIN).

In accordance with some embodiments, the inventive methods comprise application of the immunomodulator at the cutaneous and mucosal surfaces which are infected with HPV. In an embodiment, the mucosal tissue is the vaginal mucosa. In other embodiments, the cutaneous and mucosal surfaces are in the cervicovaginal tract.

As used herein, the term “subject” can mean a subject having pre-cervical cancer lesions or having an increased risk of having a cervical neoplasia and can include a patient presenting with CIN, and/or low grade squamous intraepithelial lesion (LSIL) and/or high grade squamous intraepithelial lesion (HSIL), or any other abnormal Pap smear or cytological test.

As used herein, the term “subject” can also mean a subject having an HPV infection or HPV related disease, and also includes a subject that has either been exposed to HPV or has evidence of infection with HPV of any variant strain other than HPV 16.

In accordance with one or more embodiments of the present invention, it will be understood that the types of neoplasia diagnosis which may be made, using the methods provided herein, is not necessarily limited.

As used herein, the term “treat,” as well as words stemming therefrom, includes diagnostic and preventive as well as disorder remitative treatment.

As used herein, the term “subject” refers to a human.

As used herein, the term “HPV16 negative” means the subject is infected with one or more genotypes of HPV that is not HPV16.

In accordance with some embodiments, the inventive treatment regimen comprises: a) topical administration of a sufficient amount of the immunomodulator to the site of the HPV-associated neoplasia in the HPV16 negative subject at Day 0; b) repeating administration of a sufficient amount of the immunomodulator to the site of the HPV-associated neoplasia in the subject at an interval of about 3 to 5 weeks later; and c) repeating administration, at least one additional time, of a sufficient amount of the immunomodulator to the site of the HPV-associated neoplasia in the subject at an interval of about 3 to 5 weeks later.

It will be understood by those of ordinary skill in the art, that the treatment regimen can be varied to alter the immune response of the patient. In some embodiment, the intervals between applications of the immunomodulator can be 3 weeks, 3.5 weeks, 4 weeks, 4.5 weeks, and 5 weeks or more.

In some embodiments, the administration of the immunomodulator can be repeated 2, 3, or 4 or more times. In other embodiment, the regimen can include additional administrations of immunomodulator at later periods of time, such as 3 to 6 months later.

In some embodiments, administration of the immunomodulator can be from 3 to about 15 administrations, where each administration is separated by about 1 to about 4 weeks.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of HPV-associated neoplasia in a mammal. Furthermore, the treatment or prevention provided by the inventive method can include treatment or prevention of one or more conditions or symptoms of the disease, e.g., neoplasia, being treated or prevented. Also, for purposes herein, “prevention” can encompass delaying the onset of the disease, or a symptom or condition thereof.

The term “immunomodulator,” as used herein, generally means a toll-like receptor (TLR) ligand. TLR ligands are capable of generating stronger Trm recruitment. Currently, two TLR agonists are FDA approved for use in cancer patients in addition to imiquimod, the TLR4 agonist monophosphoryl lipid A (MPL), and the TLR2/4 agonist bacillus Calmette-Guérin (BCG). Also useful in the methods of the present invention is the TLR7/8 agonist, resiquimod, which is an imidazoquinoline like imiquimod, and has been shown to have antitumor effects. TLR9 agonists are being developed for clinical application in oncology and viral infections by Pfizer, Dynavax Technologies and GlaxoSmithKline among others. It will be understood by those of skill in the art that alternative immunomodulators can be used in the inventive methods. In a specific embodiment, the immunomodulator used is imiquimod (1-(2-methylpropyl)-1H-imidazo[4,5-c]quinolin-4-amine), also known under the trade names Aldara and Zyclara, and by Mochida as Beselna. It is also referred to as R-837.

In accordance with another embodiment, the present invention provides a treatment regimen for generating an immune response against human papillomavirus (HPV)-associated neoplasia in a subject that is HPV16 negative comprising administering to the subject a composition comprising a therapeutic vaccine construct and subsequently administering to the subject an effective amount of a composition comprising an toll-like receptor 7 agonist to the site of the HPV-related disease or infection in the subject.

The attending physician will also decide the dosage of amount of immunomodulator with which to treat each individual patient, taking into consideration a variety of factors, such as age, body weight, general health, diet, sex, to be administered, route of administration, and the severity of the condition being treated. In some embodiments the dosage range, for example, is about 10 μg to about 20000 μg of an immunomodulator, such as imiquimod. In an embodiment, the dosage range of an immunomodulator, such as imiquimod, is about 50 μg to about 15000 mg. The formulations can vary with the route of administration. In some embodiments, the dosage of an immunomodulator, such as imiquimod, is about 10-15 mg per administration.

It will be understood by those of skill in the art, that the concentration of immunomodulator can be varied by weight percent in a suitable topical pharmaceutically acceptable carrier composition. For example, the immunomodulator can be present in the carrier composition in concentrations of 1%, 2%, 3%, 5%, 6%, 7%, up to, and including 10% by weight (w/w).

The immunomodulator compositions can be applied to the regions of HPV-associated neoplasia using any known acceptable means in the art, such as brushes, applicators, swabs and the like. The compositions are left in contact with the neoplastic areas for a sufficient amount of time, and in some embodiments, the compositions are removed. In some embodiments, after application to the cervical area, a tampon is applied to the area for a period of time of about 4 hours to about 10 hours, and then the tampon is removed.

In certain embodiments, the immunomodulator is administered topically, to the site of the HPV-associated neoplasia in the subject. In a specific embodiment, the immunomodulator is imiquimod, which is administered topically in a cream formulation.

Formulations suitable for vaginal administration can be presented as pessaries, tampons, creams, gels, pastes, foams, suppositories, or spray formulas containing, in addition to the active ingredient, such carriers as are known in the art to be appropriate.

An “active agent” and a “biologically active agent” are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

The biologically active agent may vary widely with the intended purpose for the composition. The term active is art-recognized and refers to any moiety that is a biologically, physiologically, or pharmacologically active substance that acts locally or systemically in a subject. Examples of biologically active agents, that may be referred to as “drugs”, are described in well-known literature references such as the Merck Index, the Physicians' Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

Specific examples of useful biologically active agents the above categories include: antineoplastics such as androgen inhibitors, alkylating agents, nitrogen mustard alkylating agents, nitrosourea alkylating agents, antimetabolites, purine analog antimetabolites, pyrimidine analog antimetabolites, hormonal antineoplastics, natural antineoplastics, antibiotic natural antineoplastics, carboplatin and cisplatin; nitrosourea alkylating antineoplastic agents, such as carmustine (BCNU); antimetabolite antineoplastic agents, such as methotrexate; pyrimidine analog antineoplastic agents, such as fluorouracil (5-FU) and gemcitabine; hormonal antineoplastics, such as goserelin, leuprolide, and tamoxifen; natural antineoplastics, such as aldesleukin, interleukin-2, docetaxel, etoposide, interferon; paclitaxel, other taxane derivatives, and tretinoin (ATRA); antibiotic natural antineoplastics, such as bleomycin, dactinomycin, daunorubicin, doxorubicin, mitomycin; vinca alkaloid natural antineoplastics, such as vinblastine and vincristine; proteasome inhibitors, such as lactacystin, bortezomib, epigallocatechin-3, salinosporamide, ONX0912, CEP-18770, MLN9708, epoxomicin, and carfilzomib; and histone deacetylase (HDAC) inhibitors, such as hydroxamic acids, benzamides, vorinostat, belinostat, panobinostat, entinostat and mocetinostat.

Other biologically active agents can include peptides, proteins, and other large molecules, such as interleukins 1 through 18, including mutants and analogues; interferons α, γ, and which may be useful for cartilage regeneration, hormone releasing hormone (LHRH) and analogues, gonadotropin releasing hormone transforming growth factor (TGF); fibroblast growth factor (FGF); tumor necrosis factor-α); nerve growth factor (NGF); growth hormone releasing factor (GHRF), epidermal growth factor (EGF), connective tissue activated osteogenic factors, fibroblast growth factor homologous factor (FGFHF); hepatocyte growth factor (HGF); insulin growth factor (IGF); invasion inhibiting factor-2 (IIF-2); bone morphogenetic proteins 1-7 (BMP 1-7); somatostatin; thymosin-α-γ-globulin; superoxide dismutase (SOD); and complement factors, and biologically active analogs, fragments, and derivatives of such factors, for example, growth factors.

It will be understood by those of ordinary skill in the art that the methods of the invention can be used in many variations of regimens, and should not be limited by any particular example.

### Examples

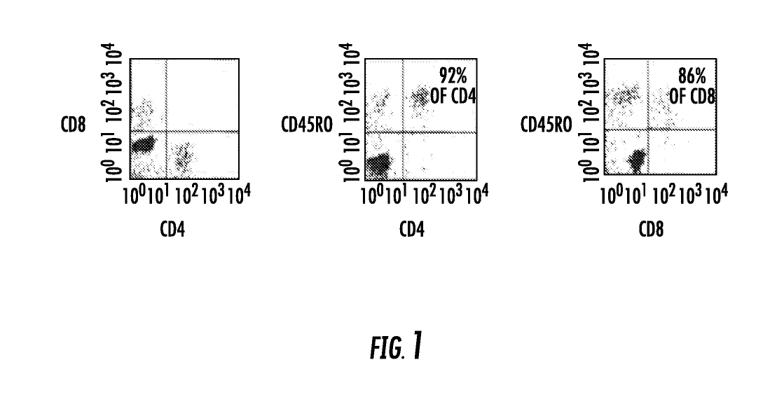

Measurement of immune responses in the cervix. The phenotype, specificity, and function of cervical mucosal immune cell populations, and their correlation with lesion outcome is assessed. Using protocols from the McElrath lab (J. Exp. Med., 1997; 185(2):293-303), who were able to isolate, expand, and characterize HIV-specific T cell subsets in cervical lymphocytes from HIV+ women, cervical lymphocytes in HPV patients were isolated and characterized. About 10⁵-10⁶ lymphocytes were isolated from cervical swabs obtained at the time of colposcopic examinations, and identified distinct phenotypic subsets using flow cytometry. Preliminary data consistently demonstrate that nearly all the T cells are CD45RO+ memory cells in both CD4+ and CD8+ lymphocytes in fresh cervical samples from all of the study patients, and they are present in a ratio of CD4+/CD8+ of 1:1 (FIG. 1).

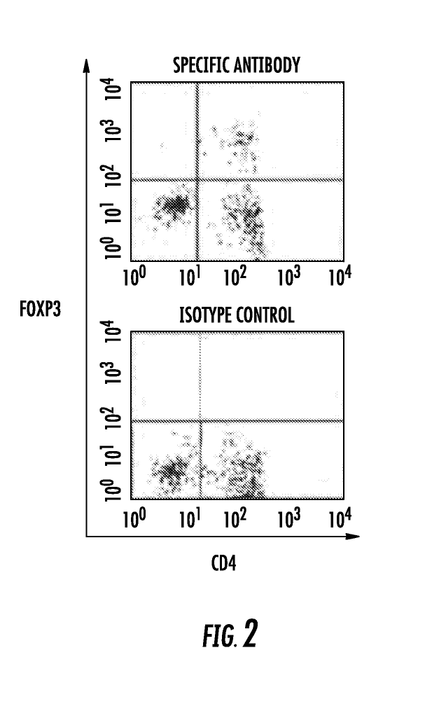

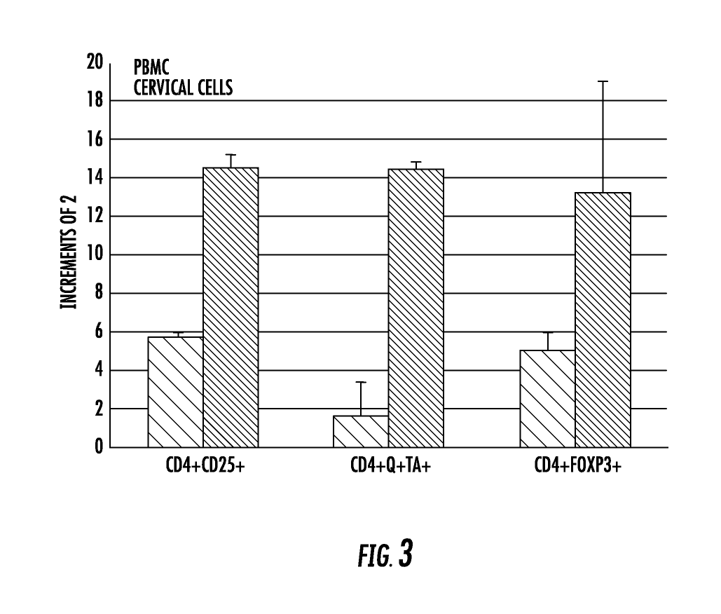

After demonstrating the feasibility of phenotyping the relatively small numbers of lymphocytes obtained from fresh cervical samples, we investigated in more detail whether T_{reg} populations are relatively enriched in lesions that fail to regress. In unvaccinated patients who have cervical dysplasia, higher frequencies of CD4+CD25+ and CD4+Foxp3+ T cells were found in the cervix than in PBLs from specimens obtained at the same time points. (FIGS. 2 and 3)

Treatment protocol. In an embodiment, patients are screened at their initial colposcopic evaluation for referral for evaluation of high grade pap smears. Immunocomptetent patients with biopsy-confirmed CIN2/3 which is HPV16 negative will be eligible for enrollment. Both treatment and control groups will be assessed for histologic diagnosis at week 15, change in cervical quantitative HPV viral load, safety, toxicity, tolerability, and for measures of local mucosal immune responses. Patients will be given imiquimod (5%) or vehicle at Day 0, week 4, and week 8 and then histological diagnosis is determined at week 15.

Imiquimod administration on the lesion at each vaccination. In an embodiment, patients received a total of three applications of imiquimod directly on the cervical lesion, one application at the time of each vaccination. One single use packet containing 250 mg of 5% imiquimod was used for each treatment. After insertion of a vaginal speculum, the cream is applied directly to the lesion by the PI, using a cotton-tipped applicator. The speculum is withdrawn, and a tampon is placed as a medication barrier. Patients will be instructed to leave the tampon in place for about 4 to 6, and no more than 10 hours. In alternative embodiments, the treatment will include vaccination with therapeutic vaccine constructs in addition to the imiquimod administration methods provided herein.

Cervical cytokine profiles. In an embodiment, endocervical brushes are used at each speculum exam to collect cervical secretions. In alternative embodiments, merocel ophthalmic sponges are used at each speculum exam to collect cervical secretions, using the method of Hildesheim et al. (Clin. Diagn. Lab. Immunol. March 2004; 11 (2):399-405). The sponge is placed gently in the os before any other manipulations are performed, and allowed to passively absorb cervical effluent for 30 seconds. In yet another embodiment, DNA collection devices can be used. At least two specimens are obtained sequentially at each exam. These are stored in individual vials at −70° C. Specimens will be analyzed in batch. The specimens are equilibrated in 300 μl PBS+0.25M NaCl, 0.1 mg of aprotinin/ml, and 0.001% sodium azide, and 12 μl/well used for the cytokine assay. Cytokine profiles from cervical specimens are quantified and matched with simultaneous peripheral blood draws using BioPlex system, which will allow quantitation of a panel of 27 cytokines in each sample. The preconfigured, standardized cytokine kit we will use includes both Th1 and Th2 panels, as well as other immunomodulatory cytokines such as GM-CSF, MIP-1a, RANTES, and VEGF. Samples will be assayed in duplicate.

Cervical lymphocyte specimens. Endocervical swabs are obtained at the time of each speculum exam and processed by the method of Musey et al. Briefly, an endocervical brush is inserted into the os of non-menstruating patients and rotated gently. Specimens visibly contaminated with blood are discarded. The brushes are placed in 3 ml RPMI with 100 U/ml penicillin, 100 mcg/ml streptomycin, and 2.5 mcg/ml amphotericin B. Specimens are transported on ice and processed within 3 hours of collection. In yet another embodiment, DNA collection devices can be used and cryopreserved and analyzed in batch at a later time. Brushes with no visible blood contamination will be agitated against the side of the tube to remove as much mucus as possible, and treated with 0.5 mM DTT for 20 minutes at 37° C. The samples will be placed over 40 μm cell strainers to remove epithelial cells. The suspension is then centrifuged (330 g, 10 min) and the pellet washed once in PBS.

Phenotypic analysis of immune cells in the cervix over the treatment interval. Flow cytometry analysis is performed to characterize surface phenotype directly ex vivo, in longitudinal cervical brush specimens in individual subjects to quantitate percentages of immune cell populations. Lymphocytes are stained with anti-human antibodies against CD4, CD8, CD25, CD45RO, Foxp3, and Lag3, followed by flow cytometry analysis. An aliquot of fresh cervical lymphocytes from matched time points is analyzed in parallel with PBL populations (J. Immunol. 2010; 185(11):7107-7114).

Cervical lymphocyte repertoire. One goal is the identification of the repertoire and function of cervical lesional lymphocytes. Since the absolute number of lymphocytes routinely obtained from these samples is low, and because the absolute number of cells that can be purified from fresh swabs is rate-limiting, two strategies are used to expand these cells. These methods can be refined to nonspecifically expand these populations, so that we can identify the repertoire of cervical lymphocyte populations. We have successfully expanded fresh lymphocyte samples from cervical swabs using anti-CD3/CD28 beads, and are able to grow populations from approximately. 5,000 cells to over 200,000 cells over a culture period of 10-14 days. However we have found that using this method, CD4+ cells are preferentially expanded over CD8+ cells (data not shown). This phenomenon has been observed by other laboratories in expanding tumor infiltrating lymphocytes (TILs). The disproportionate expansion of CD4 cells could be explained by the fact that effector CD8 cells in humans are almost entirely CD28 negative. Therefore stimulation with only CD3 and CD28 would confer a significant survival advantage to CD4 over CD8 T cells. Two strategies to expand CD8+ cells include: (1) depleting CD4+ T cells prior to culture, (2) provide additional costimulatory signals to enhance CD8+ cell proliferation.

In order to nonspecifically expand and characterize the repertoire of lesional lymphocyte populations, we use artificial antigen presenting cells obtained from the June lab. Dr. June has kindly offered the use of K562 based artificial antigen presenting cells which express ligands for the human Fc-y receptor (CD32) and human 4-1BB ligand, a costimulatory molecule. 4-1BB co-stimulation provides a survival signal critical to expansion but not initial stimulation of CD8+ T cell proliferation (Nat Biotechnol. February 2002; 20(2):143-148). IL 15 stimulates proliferation of memory CD8 T cells. IL21 synergistic effect on clonal expansion of CD8+ when delivered with IL15. Finally, the surface Fc-gamma receptors are loaded with anti-CD3 and anti-CD28 antibodies, and irradiated. Dr June's laboratory is using these aAPCs in co-culture with IL-2 to expand polyclonal populations of tumor infiltrating lymphocytes (TILs) obtained from ovarian cancer specimens, and is able to propagate and maintain diverse memory CD8+ populations.

Cervical tissue specimens Immunohistochemical analysis of immune cell populations in tissue sections over the treatment interval. Briefly, primary tissue explants were obtained from surgical resection specimens. Normal mucosa obtained on banking protocol governing acquisition of residual tissue after standard diagnostic sections had been obtained. Tissue explants were obtained from standard therapeutic resections of the cervical squamocolumnar junction, and only if the surgical pathologists had no suspicion of invasive disease on a frozen section, a 2-mm section of fresh tissue immediately adjacent to the frozen section was reserved. Primary tissue explants were cultured using the method of Clark et al. Flow cytometry analysis of cervical T cells was performed using directly conjugated monoclonal antibodies obtained from BioLegend (CD3, CD4, CD8, CD45RO, CD45RA, and L-selectin/CD62L), R&D Systems (CCR7), and the National Institutes of Health (NIH) AIDS Reagent Program (α4β7 and ACT-1). Analysis of flow cytometry samples was performed on BD Biosciences FACScan or FACSCanto instruments, and data were analyzed with FACSDiva software (version 5.1, BD Biosciences) (Sci. Transl. Med., 6:221 (2014); (J. Immunol. 2010; 185(11):7107-7114)). To quantitate the density, tissue distribution, and colocalization of molecular analytes of interest, we acquire digital images, and use image analysis software to obtain objective, quantitative measures in specifically demarcated regions of interest (J. Immunol. 2010; 185(11):7107-7114)..

Statistical analysis. All data are expressed as mean±S.E. where indicated. Comparisons between individual data points for intracellular cytokine staining with flow cytometric analysis and tumor treatment were made using Student's t-test. Binary endpoints, such as histology, were compared using paired t-tests. Comparison of treatment groups was analyzed using Wilcoxon matched pairs signed rank tests.

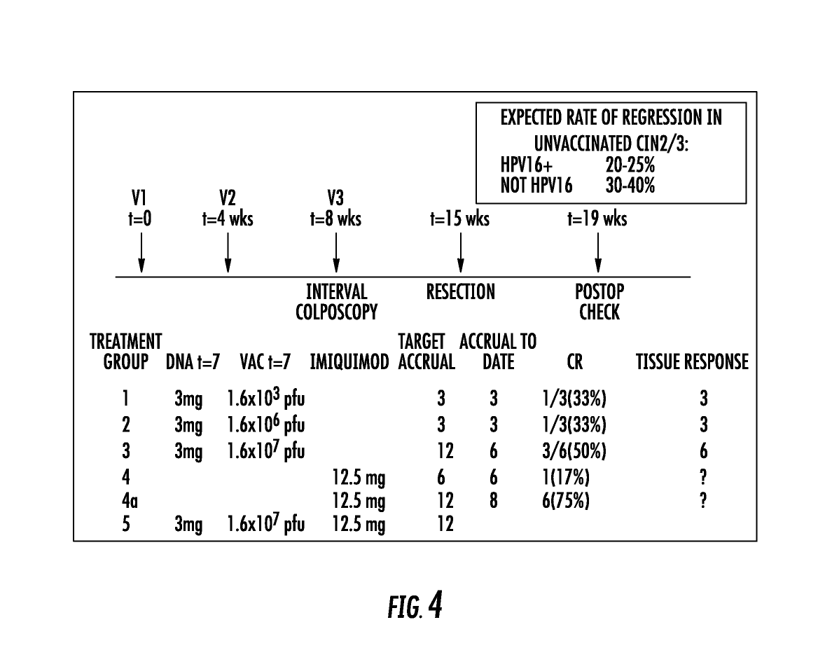

Clinical trial results. In an investigator-initiated IND, supported by NIH funding, a clinical trial testing feasibility, tolerability, and efficacy of topical application of the immune modulator imiquimod, a TLR7/8 agonist on CIN 2/3 lesions was performed. Subjects in this cohort underwent direct application on the cervical lesion, of one packet of imiquimod (12.5 mg of imiquimod formulated in 250 mg of cream per packet) a total of three times, at four-week intervals. After application by the healthcare provider, a tampon was inserted, and left in place for 4 to 6 hours. Subjects were monitored for mucosal irritation. Assessment of lesion status was performed eight weeks after the third application of imiquimod. To date, rates of regression in subjects with CIN2/3 lesions associated with HPV genotypes other than HPV16 are significantly greater (10/16; 62.5% regression) than what was expected in subjects who did not get this treatment regimen. These encouraging clinical results have prompted us to expand the clinical trial cohort (FIG. 4).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A treatment regimen for generating an immune response against human papillomavirus (HPV)-associated neoplasia in a subject that is HPV16 negative, comprising administering to the subject an effective amount of a composition comprising an immunomodulator to the site of the HPV-associated neoplasia in the subject.

2. The treatment regimen of claim 1, wherein the immunomodulator is a toll-like receptor (TLR) ligand.

3. The treatment regimen of claim 2, wherein the TLR ligand is a TLR agonist.

4. The treatment regimen of claim 3, wherein the TLR agonist is selected from the group consisting of imiquimod, monophosphoryl lipid A (MPL), bacillus Calmette-Guérin (BCG) and resiquimod.

5. The treatment regimen of claim 1, wherein the HPV-associated neoplasia is selected from the group consisting of cervical intraepithelial neoplasia (CIN), vulvar intraepithelial neoplasia (VIN), and vaginal intraepithelial neoplasia (VAIN).

6. The treatment regimen of claim 1, wherein regimen comprises:
a) topical administration of a sufficient amount of the immunomodulator to the site of the HPV-associated neoplasia in the subject at Day 0;
b) repeating administration of a sufficient amount of the immunomodulator to the site of the HPV-associated neoplasia in the subject at an interval of about 3 to 5 weeks later; and
c) repeating administration, at least one additional time, of a sufficient amount of the immunomodulator to the site of the HPV-associated neoplasia in the subject at an interval of about 3 to 5 weeks later.

7. The treatment regimen of claim 6, wherein the immunomodulator is imiquimod.

8. The treatment regimen of claim 7, wherein the imiquimod concentration is between about 1% w/w to about 10% w/w.

9. The treatment regimen of claim 8, wherein the imiquimod concentration is 5% w/w.

10. The treatment regimen of claim 1, wherein the treatment regimen further comprises administering to the subject at least one additional biologically active agent.
